(19) United States
(12) Patent Application Publication
(10) Pub. No.: US 2020/0017876 A1
(43) Pub. Date: Jan. 16, 2020

(54) COMPOSITION FOR INCREASING EXPRESSION LEVEL OF INTERFERON IN ANIMAL CELLS USING PLANT-DERIVED DNA DEMETHYLASE, ANTIVIRAL COMPOSITION, AND METHOD USING SAME

(71) Applicant: SEOUL NATIONAL UNIVERSITY R&DB FOUNDATION, Seoul (KR)

(72) Inventors: Jin Hoe HUH, Seoul (KR); Young Geun MOK, Seoul (KR)

(73) Assignee: SEOUL NATIONAL UNIVERSITY R&DB FOUNDATION, Seoul (KR)

(21) Appl. No.: 16/495,559

(22) PCT Filed: Jul. 27, 2018
(86) PCT No.: PCT/KR2018/008580

§ 371 Date: Sep. 19, 2019

(30) Foreign Application Priority Data
Jul. 28, 2017 (KR) 10-2017-0096227

(51) Int. Cl. C12N 15/85; C07K 14/56; C07K 14/565; C07K 14/57

(52) U.S. Cl. CPC C12N 15/85; C07K 14/57; C07K 14/565; C07K 14/56

(57) ABSTRACT

Provided are a composition or a kit for increasing the expression level of interferon in animal cells, an antiviral composition, and a method for increasing the expression level of interferon in animal cells using the same. Accordingly, by inducing the expression of DME in animal cells, the expression of IFNβ is increased without cell division, and thus it is possible to induce an antiviral reaction within a short time.

### FIELD

Provided are a composition or a kit for increasing an expression level of interferon in animal cells using plant-derived DNA demethylase, an antiviral composition, and a method of increasing an expression level of interferon in animal cells using the same.

### BACKGROUND ART

DNA methylation is one of the major epigenetic mechanisms that regulate chromatin structure and gene expression. Generally, DNA hypermethylation induces chromatin condensation and suppresses gene expression, whereas DNA demethylation induces chromatin decondensation and gene expression. DNA methylation in eukaryotes generally refers to conversion of cytosine, which is one of the DNA bases, to 5-methylcytosine (5 mC) by DNA methyltransferase (DNMT). DNA demethylation refers to conversion of 5-methylcytosine to cytosine, and passive or active demethylation occurs. In animals, active demethylation involves oxidation of 5 mC to 5-hydroxymethylcytosine (5hmC) and sequential conversion to 5-formylcytosine (5fC) and 5-carboxylcytosine (5caC) by ten-eleven translocation (TET) protein, which is a DNA hydroxylase, followed by excision by a mismatch glycosylase such as thymine glycosylase. In plants, active demethylation of 5 mC involves direct excision of 5 mC from DNA and consequent replacement with cytosine via base excision repair mediated by DEMETER (DME), which is a 5 mC DNA glycosylase (Choi et al., Cell, vol. 110, pp. 33-42, Jul. 12, 2002; Gehring et al., Cell, vol. 124, p. 495-506, Feb. 10, 2006). The DME gene family of plants has been known to encode the only protein having the ability to directly excise 5 mC in eukaryotes.

Since abnormal DNA methylation in humans is known to act as a cause of major diseases including cancers, a DNA demethylating agent may be used as a therapeutic agent. For example, DNA demethylating agents such as 5-azacitidine (Vidaza®), 5-aza-2′-deoxycytidine (Decitabine, Dacogen®), etc. are used as therapeutic agents for cancers or myelodysplastic syndrome (MSD).

Accordingly, it is necessary to develop a method of using DME to prevent and treat various diseases by expressing DME, which is a plant-derived DNA demethylation gene, to induce genomic demethylation in animal cells and to analyze cell signaling therefrom.

### DESCRIPTION OF EMBODIMENTS

### Technical Problem

Provided is a composition for increasing an expression level of interferon in animal cells.

Provided is an antiviral composition for increasing an expression level of interferon in animal cells.

Provided is a kit for increasing an expression level of interferon in animal cells.

Provided is a method of increasing an expression level of interferon in animal cells.

### Solution to Problem

An aspect provides a composition for increasing an expression level of interferon in animal cells, the composition including a plant-derived polypeptide having catalytic activity in DNA demethylation or including a polynucleotide encoding the polypeptide.

The plant-derived polypeptide may be Arabidopsis thaliana-derived DNA demethylase.

The polypeptide may be DNA demethylase such as a DEMETER (DME) or a variant thereof which has 5 mC DNA glycosylase activity. The DME may be a polypeptide including an amino acid sequence of SEQ ID NO: 1, or a polypeptide having about 99% or more, about 95% or more, about 90% or more, about 80% or more, or about 70% or more of sequence identity thereto. The DME may include an amino acid sequence of Genbank Accession No. AAM77215. The DEMETER variant may have a deletion of an amino acid sequence at positions 1 to 677 from the N-terminus of the amino acid sequence of the DME of SEQ ID NO: 1. The DEMETER variant may consist of an amino acid sequence of SEQ ID NO: 2.

The polypeptide may be a demethylase that excises methylated cytosine from DNA. The polypeptide may be a DNA glycosylase that excises methylated cytosine from DNA by hydrolyzing a glycosidic bond of a nucleotide. The methylated cytosine may be 5-methylcytosine (5 mC). The polypeptide may mediate base excision repair that excises a base of 5-methylcytosine from 5-methylcytosine nucleoside.

The polynucleotide may be included in an expression vector. The expression vector may be a vector capable of expressing a protein in animal cells.

The animal cell may be a mammalian cell derived from a human, a cow, a horse, a pig, a dog, a sheep, a goat, a cat, or a mouse. The animal cell may be a renal cell, an immune cell, a cancer cell, a heart cell, a nerve cell, a beta cell (8-cell), a stem cell, or a fibroblast cell.

The interferon (IFN), which is a protein produced in immune cells of vertebrates, refers to a cytokine in response to foreign agents such as viruses, bacteria, parasites, tumor cells, etc. The interferon may induce immune responses that suppress viral proliferation in cells. The interferon may activate natural killer cells, macrophage cells, dendritic cells, etc. The interferon may be type 1 interferon, type 2 interferon, or type 3 interferon. The interferon may be selected from the group consisting of interferon-alpha (IFN-α), interferon-beta (IFN-β), interferon-gamma (IFN-γ), interferon kappa (IFN-κ), interferon-delta (IFN-δ), interferon epsilon (IFN-ε), interferon tau (IFN-τ), interferon omega (IFN-w), and interferon zeta (IFN-ζ).

The increase of the expression level of interferon may be an increase in an amount of mRNA encoding the interferon, a lifespan of mRNA, an amount of the interferon protein, a lifespan of the interferon protein, or a release amount of the interferon protein during transcription, translation, or post-translational processing of the interferon. The increase of the expression level of interferon may be an increase of the expression level of interferon, as compared with that in a negative control group. The negative control group may be an animal cell not including the plant-derived polypeptide having the catalytic activity in DNA demethylation or not including the polynucleotide encoding the polypeptide.

The composition may be for in vitro, in vivo, or ex vivo administration.

Another aspect provides an antiviral composition including the plant-derived polypeptide having the catalytic activity in DNA demethylation or including the polynucleotide encoding the polypeptide.

The plant-derived polypeptide, polynucleotide, and DNA demethylation are the same as described above.

The antiviral composition may be a pharmaceutical composition which may be used in suppressing cell proliferation and preventing or treating viral infection or diseases caused by the viral infection. The term “preventing” refers to all of the actions by which viral infection is restrained or retarded by administering the pharmaceutical composition. The term “treating” refers to all of the actions by which symptoms caused by viral infection have taken a turn for the better or been modified favorably by administering the pharmaceutical composition.

The composition may include a pharmaceutically acceptable carrier. The carrier is used as the meaning including an excipient, a diluent, or an adjuvant. The carrier may be, for example, selected from the group consisting of lactose, dextrose, sucrose, sorbitol, mannitol, xylitol, erythritol, maltitol, starch, acacia gum, alginate, gelatin, calcium phosphate, calcium silicate, cellulose, methylcellulose, polyvinyl pyrrolidone, water, physiological saline, a buffer such as PBS, methylhydroxy benzoate, propylhydroxy benzoate, talc, magnesium stearate, and mineral oil. The composition may include a filler, an anti-coagulant, a lubricant, a wetting agent, a flavoring agent, an emulsifying agent, a preservative, or a combination thereof.

The composition may be prepared into any formulation according to a common method. The composition may be prepared as, for example, a formulation for oral administration (e.g., a powder, a tablet, a capsule, a syrup, a pill, or granules), or a formulation for parenteral administration (e.g., an injectable formulation). The composition may also be prepared as a systemic formulation or a topical formulation.

The composition may further include another antiviral agent or antimicrobial agent.

Still another aspect provides a kit for increasing the expression level of interferon in animal cells, the kit including the plant-derived polypeptide having the catalytic activity in DNA demethylation or including the polynucleotide encoding the polypeptide.

The plant-derived polypeptide, polynucleotide, DNA demethylation, animal cell, interferon, and increase of the expression level of interferon are the same as described above.

The kit may further include a substance for introducing the polypeptide or polynucleotide into the animal cells. The kit may include reagents needed for transformation, transduction, transfection, or injection. The kit may include, for example, lipofectamine.

Still another aspect provides a method of increasing the expression level of interferon in animal cells, the method including introducing the polynucleotide encoding the plant-derived polypeptide having the catalytic activity in DNA demethylation into the animal cells by incubating the polynucleotide with the animal cells.

The plant-derived polypeptide, polynucleotide, DNA demethylation, animal cell, interferon, and increase of the expression level of interferon are the same as described above.

The method may include introducing the polynucleotide into the animal cells by incubating the polynucleotide with the animal cells. The introducing may be transformation, transduction, transfection, or injection.

The method may induce expression of interferon genes through DNA demethylation of the animal cells.

The method may induce anti-viral responses in cells in vitro or in a subject. When anti-viral responses may be induced by the method, the method may be used in preventing or treating viral infection of a subject. The subject may be a human, a cow, a horse, a pig, a dog, a sheep, a goat, or a cat. The subject may be a subject who has had virial infection or has a high risk of viral infection. The administration may be oral or parenteral administration. The administration may be performed via, for example, oral, transdermal, subcutaneous, rectal, intravenous, intraarterial, intraperitoneal, intramuscular, intrasternal, topical, intranasal, intratracheal, or intradermal route. The polynucleotide may be systemically or topically administered alone or in combination with another pharmaceutically active compounds. An administration dose of the polynucleotide may vary depending on a patient's conditions and body weight, severity of the disease, a type of the drug, administration route and period, but may be appropriately selected by those skilled in the art. For example, the administration dose may be within the range of about 0.001 mg/kg to about 100 mg/kg, about 0.01 mg/kg to about 10 mg/kg, or about 0.1 mg/kg to about 1 mg/kg per adult. The administration may be performed once a day, several times a day, once a week, once every two weeks, once every three weeks, once every four weeks, or once a year.

### Advantageous Effects of Disclosure

According to a composition or a kit for increasing an expression level of interferon in animal cells, an antiviral composition, and a method of increasing the expression level of interferon in animal cells using the same, DME expression may be induced in the animal cells to increase an expression level of IFNβ by DME without cell division, thereby inducing anti-viral responses in a short period of time.

### BRIEF DESCRIPTION OF THE DRAWINGS

FIG. 1A is a schematic representation of the structure of a DMEΔ polypeptide, and FIGS. 1B and 1C show confocal laser microscope images of animal cells transfected with GFP or a GFP-DME expression vector pCDNA3.1, and results of an in vitro 5-methylcytosine excision experiment, respectively;

FIG. 2A is a graph showing the number of DME-expressing cells (per ml) with respect to incubation time of the cells, FIG. 2B is a graph showing the number of cells (per ml) with respect to incubation time of the cells in the presence of DME and 5-azacytidine, and FIG. 2C shows graphs showing a cell division cycle of DME-expressing cells (left: GFP-transfected cells, right: DMEΔ-transfected cells);

FIGS. 3A and 3B are images showing results of TUNEL assays and a graph showing results of analyzing TUNEL-positive cells (%) by flow cytometry, respectively;

FIGS. 4A to 4C are graphs showing results of real-time polymerase chain reaction of interferon-stimulated genes (ISGs), genes involved in cell division, and heat shock protein genes in DME-transfected cells, respectively; and

FIG. 5 shows an immunoblotting image showing expression levels of IFNβ in DME-transfected cells according to time after transfection and a graph showing expression fold change, respectively.

### MODE OF DISCLOSURE

Hereinafter, the present disclosure will be described in more detail with reference to exemplary embodiments. However, these exemplary embodiments are for illustrating one or more specific embodiments, and the scope of the present disclosure is not intended to be limited thereby.

Experimental Method

1. Transformation

A cytomegalovirus nuclear localization signal (NLS) was prepared, and inserted into a pEGFP-C1 vector (Clontech) at Bgl II and Sal I sites.

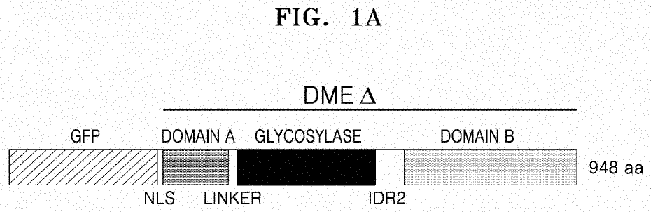

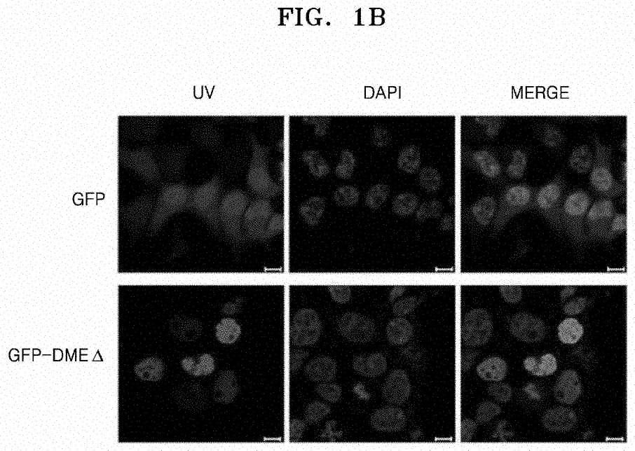

As described in Mok, Y. G. et al., Proc. Natl. Acad. Sci. USA, 2010, vol. 107, pp. 19225-19230, a DMEΔN677ΔIDR1::Ink fragment (hereinafter, referred to as DMEΔ) was prepared. In detail, DME was modified such that from Arabidopsis thaliana-derived DME having 5-methylcytosine excision activity (SEQ ID NO: 1; Genbank Accession No. AAM77215.1; Choi et al., Cell, vol. 110, pp. 33-42, Jul. 12, 2002), a site unnecessary for the activity was removed, and domain A, glycosylase domain, and domain B were included. Removal of catalytically unnecessary domains increases protein expression, solubility and stability (U.S. Pat. No. 8,951,769). Therefore, DMEΔ (SEQ ID NO: 2) was prepared as a DME variant in which the N-terminus and an interdomain region between domain A and glycosylase domain were removed for better stability. In FIG. 1A, “GFP” represents a green fluorescence protein, “NLS” represents a nuclear localization signal (SEQ ID NO: 3), “linker” or “Ink” represents a linker sequence of N-AGSSGNGSSGNG-C(SEQ ID NO: 4), and “IDR2” represents an interdomain region 2.

DMEΔ was inserted into the Sal I and Barn HI sites of pEGFP-C1 vector with NLS to prepare pEGFP-NLS-DMEΔ. The EGFP-NLS-DMEΔ fragment was amplified, and then cloned into a pCDNA3.1/hygro/lacZ vector to prepare pCDAN3.1-GFP-NLS-DMEΔ. As a negative control group, pCDAN3.1-GFP was used.

2. Transfection

To transfect animal cells with the recombinant vector including the plant-derived DME gene, HEK-293T cells derived from human embryonic kidney were prepared at a density of 5×10⁵ cells in a 60 mm cell culture plate 24 hours before transfection. Each 10 μg of GFP and GFP-DME each cloned into the pCDNA3.1 vector was prepared and transfected into HEK-293T cells using lipofectamine 2000 (Invitrogen, USA).

3. Test of 5-Methylcytosine Excision of GFP-DME

48 hours after transfection, only the green fluorescence protein-positive cells were sorted from the GFP or GFP-DME-treated cells using FACS Aria III, and thus transfected cells were harvested. The harvested cells were collected by centrifugation, and resuspended in a cell lysis buffer (50 mM Tis-HCl, pH 7.4, 100 mM NaCl, 10% (v/v) glycerol, 0.1 mM dithiothreitol (DTT), and a protease inhibitor cocktail tablet (Roche)). A tube containing the cells was put on ice, and cells were lysed by sonication. After cell lysis, centrifugation was performed to collect a supernatant containing water-soluble proteins, and this whole cell extract was used in a subsequent experiment.

A 35 mer-oligonucleotide having 5-methylcytosine of the following nucleotide sequence was labeled with radioisotopes using T4 polynucleotide kinase:

(SEQ ID NO: 5)
5′-CTATACCTCCTCAACTC[5mC]GGTCACCGTCTCCGGCG-3′

10 μg of the whole cell extract was put into the radioisotope-labeled oligonucleotide, and incubated at 37° C. for 4 hours.

4. Cell Proliferation Assay

Every 24 hours from 24 hours to 96 hours after transfection of GFP or GFP-DME into HEK-293T, the number of cells was counted using a hemocytometer. The cells were cultured while replacing with a fresh medium containing 200 μg/mL of hygromycin B every 24 hours from 24 hours after transfection.

5. Analysis of Cell Division Cycle

48 hours after transfection of GFP or GFP-DME into HEK-293T, cells were harvested. The harvested cells were resuspended in 70% (v/v) cold ethanol and fixed with agitation at 4° C. overnight. The fixed cells were harvested using a centrifuge, and then resuspended in 0.5 mL of a buffer (10 mM HEPES, pH 7.4, 140 mM NaCl, 2.5 mM CaCl₂) and 10 μL RNase A (80 μg/ml, Sigma, USA)) and then 25 μL of 50 μg/mL concentration of propidium iodide (PI) was added thereto, followed by stirring for 30 minutes in the dark at room temperature. Thereafter, the PI-stained cells were subjected to analysis on a FACS Calibur, and cell division cycle was examined.

6. Terminal Deoxynucleotidyl Transferase dUTP Nick End Labeling (TUNEL) Assay

48 hours after transfection of GFP or GFP-DME into HEK-293T, cells were harvested. The harvested cells were resuspended in 70% cold ethanol and fixed with agitation at 4° C. overnight. The fixed cells were harvested using a centrifuge, and then resuspended in 0.1 mL of a buffer (5× reaction buffer, 25 mM CoCl₂, 0.02 mM BrdUTP, terminal deoxynucleotidyl transferase (TdT, 400 Unit)) and incubated for 1 hour at 37° C. under high humidity with shaking every 15 minutes.

The cells were washed with PBS buffer, and reacted with a 1:50 dilution buffer (5% (w/v) BSA, 0.3% (v/v) Triton X-100 in PBS) of BrdUTP alexa 594-conjugated antibody (Life Technologies) for 30 minutes in the dark at room temperature. After reaction, RNase was added thereto, and further allowed to react under the same conditions for 30 minutes. Then, the cells were washed with PBS buffer and analyzed with FACS Calibur.

7. Immunoblotting

Cells were lysed in a lysis buffer (50 mM Tis-HCl, pH 8.0, 150 mM NaCl, 0.5% (v/v) Nonidet P-40 and, and a protease inhibitor cocktail tablet (Roche)) to obtain total proteins. The obtained total proteins were electrophoresed on a SDS-polyacrylamide, followed by immunoblotting. Primary antibodies used in the immunoblotting were anti-IFNβ antibody (Abcam) and anti-β-actin antibody (Santa Cruz Biotechnology).

### Example 1. Examination of 5-Methylcytosine Excision after Expressing Plant DNA Demethylase DME in HEK-293T Animal Cells

GFP-DME was cloned into a pCDNA3.1 vector, which was prepared for transfection into animal cells. GFP to be used as a control group was cloned into a pCDNA3.1 vector, and then transfected into HEK-293T cells using lipofectamine 2000. To examine expression of GFP or GFP-DME in animal cells and intracellular localization of GFP-DME, a confocal laser microscope was used to observe the transfected cells. The in vitro experimental results of analyzing plant-derived DME expression and DNA-demethylating activity in the transfected animal cells are shown in FIGS. 1B and 1C, respectively. In FIG. 1B, a green color represents GFP or GFP-DME, and a blue color represents 4′,6-diamidino-2-phenylindole (DAPI)-stained nucleus. In FIG. 1C, “S” represents substrates, and “P” represents 5-methylcytosine excision products.

As shown in FIG. 1B, expression of GFP or GFP-DME in animal cells was confirmed. GFP was evenly localized throughout the cytoplasm, and GFP-DME was localized in the nucleus of animal cells. Therefore, it was confirmed that the plant demethylase DME was expressed in the nucleus of animal cells.

Further, as shown in FIG. 1C, a whole cell extract of the transfected cells was used to analyze 5-methylcytosine excision of radio-labelled oligonucleotide, and as a result, it was confirmed that GFP-DME expressed in animal cells effectively excised 5-methylcytosine.

### Example 2. Change in Cells by DME Expression

A DME gene is a gene that is found only in plants but not in animals. Since intracellular change by introduction of a foreign gene is predicted, cell proliferation and division patterns of transfected animal cells were analyzed.

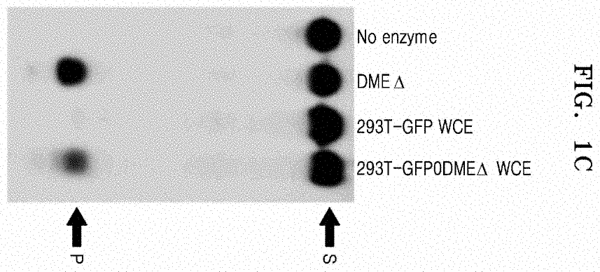

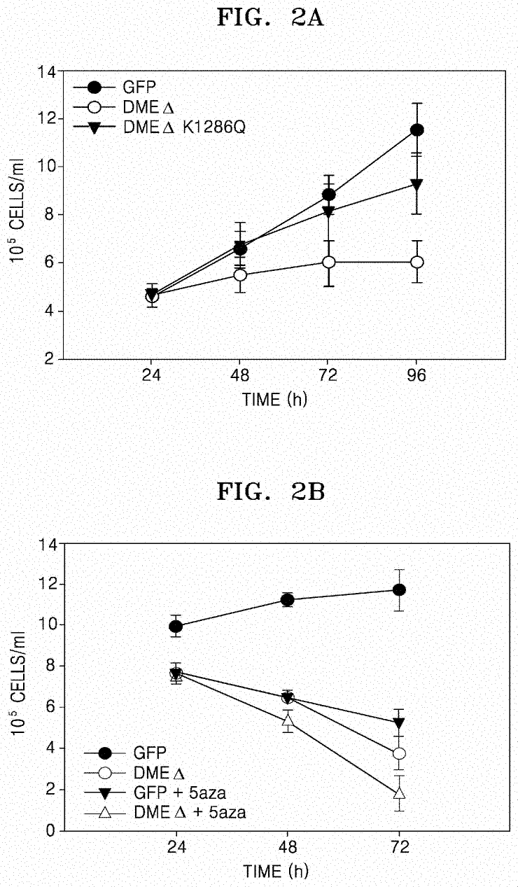

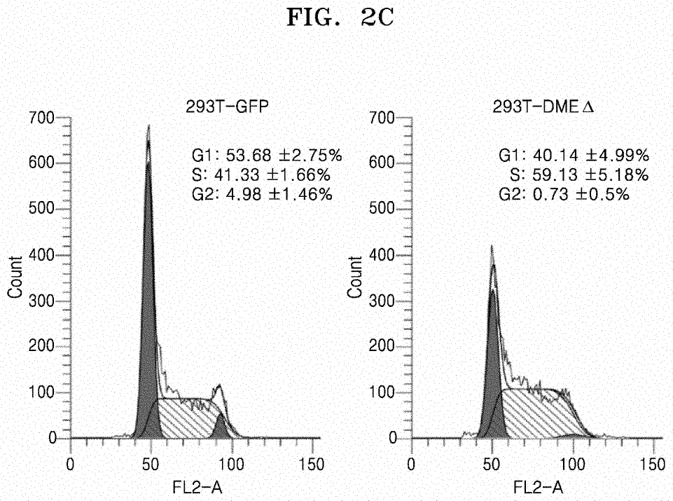

The number of DME-expressing cells with respect to incubation time is shown in FIG. 2A. DMEΔ K1286Q has no demethylating function due to a catalytic mutation of DME. As shown in FIG. 2A, the number of the GFP-transfected cells increased over the incubation time, indicating normal proliferation. In contrast, proliferation of the GFP-DME-transfected cells was confirmed to be suppressed. In particular, since DME K1286Q is a catalytic mutation of DME having no normal 5-methylcytosine excision function, the number of cells tended to increase over the incubation time, like the GFP-transfected cells, indicating that cell proliferation was suppressed by transfection with DME having the normal function.

Further, cell proliferation patterns by treatment with 5-azacytidine which is a DNA methyltransferase inhibitor are shown in FIG. 2B. As shown in FIG. 2B, it was confirmed that cell proliferation of GFP-DME-expressing cells was further reduced by treatment with 5-azacytidine.

Cell division cycles were analyzed, and results are shown in FIG. 2C. As shown in FIG. 2C, GFP-expressing cells showed normal division (left graph), but GFP-DME-expressing cells showed arrest in S phase of the cell division cycle, during which DNA replication occurs (right graph).

### Example 3. DNA Damage by DNA Demethylation

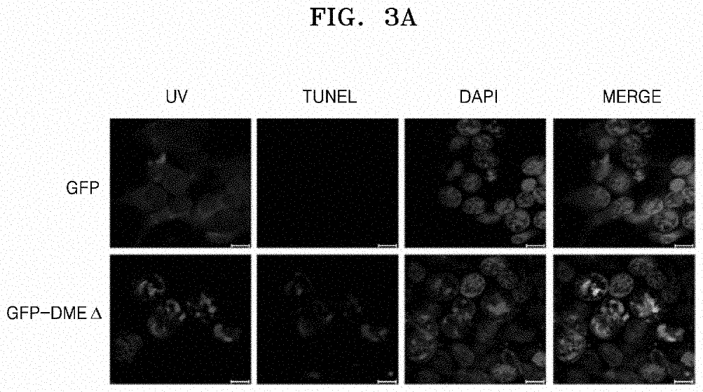

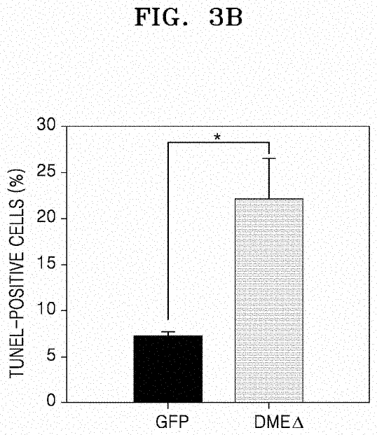

The plant-derived DME directly recognizes and excises 5-methylcytosine. Further, DNA damage may occur due to excessive excision of 5-methylcytosine, and thus terminal deoxynucleotidyl transferase dUTP nick end labeling (TUNEL) assay was performed to examine damaged DNA in DME-transfected cells. DNA damage caused by excessive DNA demethylation was analyzed through antigen-antibody reactions by confocal laser microscopy and flow cytometry, and results are shown in FIGS. 3A and 3B, respectively (in FIG. 3A, scale bar: 10 μm). As shown in FIGS. 3A and 3B, about 3 times or more of DNA damage was observed in DME-expressing cells.

### Example 4. Changes of Gene Expression by DNA Demethylation

The plant-derived DNA demethylase excises 5-methylcytosine from DNA to induce gene expression. Therefore, changes of gene expression patterns in DME-transfected cells were analyzed. The analysis was performed using a human genome U133 plus 2.0 array platform of Affymatrix. A list of genes of which expressions are changed in the DME-expressing cells is shown in Table 1 below.

TABLE 1

| Protein group and | | | | Fold | |
|---|---|---|---|---|---|
| accession no. | Gene | Symbol | Locus | change | p-value |
| Cell cycle components | | | | | |
| NM_000389 | cyclin-dependent kinase inhibitor | CDKN1A | chr6p21.2 | 2.82 | 0.031 |
| | 1A (p21, Cip1) | | | | |
| NM_001135733 | tumor protein p53 inducible nuclear | TP53INP1 | chr8q22 | 2.75 | 0.002 |
| | protein 1 | | | | |
| NM_152562 | cell division cycle associated 2 | CDCA2 | chr8p21.2 | 2.00 | 0.001 |
| NM_031299 | cell division cycle associated 3 | CDCA3 | chr12p13 | 1.96 | 0.0004 |
| NM_001790 | cell division cycle 25 homolog C | CDC25C | chr5q31 | 1.90 | 0.001 |
| | (S. pombe) | | | | |
| NM_018101 | cell division cycle associated 8 | CDCA8 | chr1p34.3 | 1.82 | 0.00006 |
| NM_001170406 | Cyclin-dependent kinase 1 | CDK1 | chr10q21.1 | 1.58 | 0.0005 |
| NM_001130851 | cyclin-dependent kinase inhibitor 3 | CDKN3 | chr14q22 | 1.56 | 0.001 |
| NM_031966 | cyclin B1 | CCNB1 | chr5q12 | 1.50 | 0.001 |
| NM_002467 | v-mye myelocytomatosis viral | MYC | chr8q24.21 | −1.60 | 0.001 |
| | oncogene homolog (avian) | | | | |
| NM_053056 | cydin D1 | CCND1 | chr11q13 | −1.84 | 0.001 |
| Interferon genes | | | | | |
| NM_006820 | interferon-induced protein 44-like | IFI44L | chr1p31.1 | 14.62 | 0.0005 |
| NM_001548 | interferon-induced protein with | IFIT1 | chr10q23.31 | 5.93 | 0.0001 |
| | tetratricopeptide repeats 1 | | | | |
| NM_001031683 | interferon-induced protein with | IFIT3 | chr10q24 | 5.15 | 0.0002 |
| | tetratricopeptide repeats 3 | | | | |
| NM_001547 | interferon-induced protein with | IFIT2 | chr10q23.31 | 4.50 | 0.003 |
| | tetratricopeptide repeats 2 | | | | |
| NM_006074 | tripartite motif-containing 22 | TRIM22 | chr11p15 | 3.97 | 0.002 |
| NM_022873 | interferon, alpha-inducible protein 6 | IFI6 | chr1p35 | 3.88 | 0.0004 |
| NM_001572 | interferon regulatory factor 7 | IRF7 | chr11p15.5 | 2.95 | 0.001 |
| NM_005101 | ISG15 ubiquitin-like modifier | ISG15 | chr1p36.33 | 2.44 | 0.0004 |
| NM_014314 | DEAD (Asp-Glu-Ala-Asp) box | DDX58 | chr9p12 | 2.35 | 0.0004 |
| | polypeptide 58 | | | | |
| NM_006187 | 2′-5′-oligoadenylate synthetase | OAS3 | chr12q24.2 | 2.29 | 0.002 |
| | 3, 100 kDa | | | | |
| NM_006435 | interferon induced transmembrane | IFITM2 | chr11p15.5 | 2.10 | 0.001 |
| | protein 2 (1-8D) | | | | |
| Heat shock proteins | | | | | |
| NM_002155 | heat shock 70 kDa protein 6 | HSPA6 | chr1q23 | 23.30 | 0.00006 |
| | (HSP70B′) | | | | |
| NM_005345 | heat shock 70 kDa protein 1A | HSPA1A | chr6p21.3 | 8.92 | 0.0001 |
| NM_006145 | DnaJ (Hsp40) homolog, subfamily | DNAJB1 | chr19p13.2 | 5.63 | 0.00009 |
| | B, member 1 | | | | |
| NM_021979 | heat shock 70 kDa protein 2 | HSPA2 | chr14q24.1 | 3.71 | 0.0007 |
| NM_001540 | heat shock 27 kDa protein 1 | HSPB1 | chr7q11.23 | 3.50 | 0.0001 |
| NM_007034 | DnaJ (Hsp40) homolog, subfamily | DNAJB4 | chr1p31.1 | 1.94 | 0.0003 |
| | B, member 4 | | | | |
| NM_006644 | heat shock 105 kDa/110 kDa | HSPH1 | chr13q12.3 | 1.91 | 0.00003 |
| | protein 1 | | | | |
| NM_002154 | heat shock 70 kDa protein 4 | HSPA4 | chr5q31.1 | −1.53 | 0.002 |

### Example 5. Increase of Interferon-Stimulated Genes Having Antiviral Function

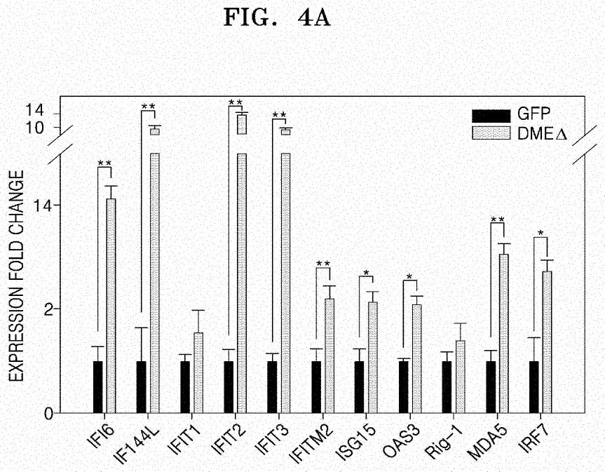

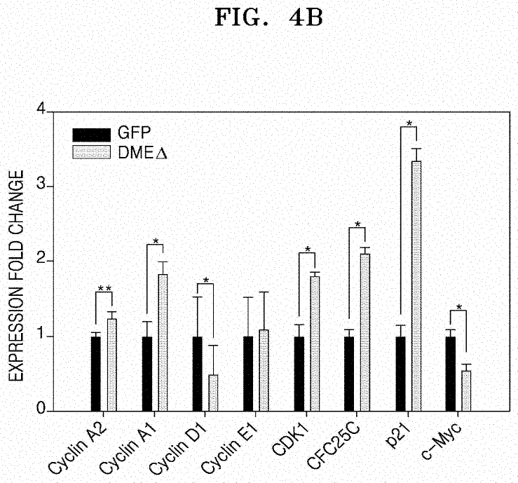

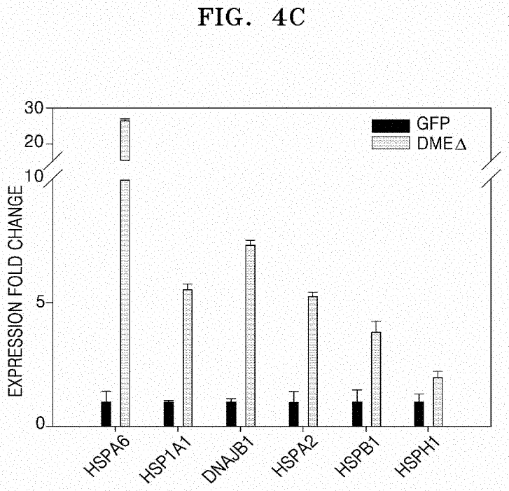

The results of analyzing changes of gene expression by DME showed that expressions of interferon (IFN)-stimulated genes having antiviral function were increased in DME-expressing cells (Table 1). Based on the above results, real-time polymerase chain reaction was performed to examine the expression patterns of major genes. Expression fold of interferon-stimulated genes (ISGs), expression fold of genes involved in cell division, and expression fold of genes of heat shock proteins are shown in FIGS. 4A to 4C (analyzed by a paired sample t-test, *: p<0.05, **: p<0.005), respectively.

As shown in FIGS. 4A to 4C, expressions of interferon-stimulated genes, cell cycle-related genes, and heat shock proteins which are molecular chaperons were increased by DME expression. In particular, IFITs are proteins which are highly upregulated upon viral infection and known to directly bind to viral RNA to suppress viral RNA translation. As shown in FIG. 4A, many different types of IFITs were significantly upregulated in GFP-DMEΔ-transfected HEK-293T, indicating that antiviral response may be induced by DME expression.

### Example 6. Induction of Antiviral Response by DME Expression

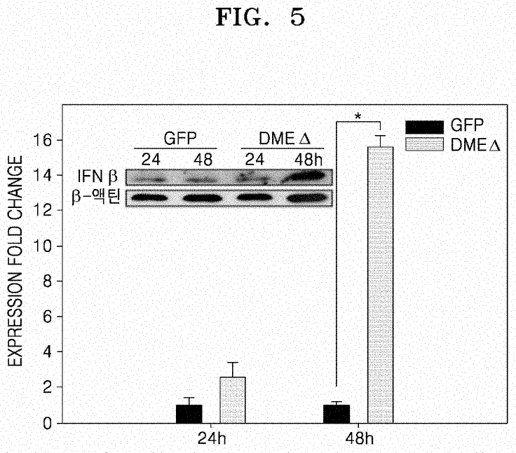

IFNs are cell signaling proteins secreted from infected cells and induce cell-intrinsic antimicrobial states. Since expression of interferon-stimulated genes is increased by DME, it was examined whether IFNβ expression was changed in DMEΔ-transfected HEK-293T cells. 24 hours and 48 hours after transfection of GFP or GFP-DMEΔ, protein expression patterns were examined by immunoblotting. Immunoblotting image and band strength were analyzed, and a graph showing expression fold change is shown in FIG. 5 (*: p<0.05).

As shown in FIG. 5, GFP-DMEΔ-transfected HEK-293T cells were found to produce a high level of IFNβ after about 48 hours. IFNβ is a protein having antiviral activity, which is involved in the innate immune response, and thus antiviral response may be triggered by inducing DME expression in animal cells. Furthermore, as shown in FIG. 2C, cell proliferation was suppressed by DME expression, and thus antiviral response may be induced without cell division within a short period of time by increasing IFNβ expression.

1. A composition for increasing an expression level of interferon in animal cells, the composition comprising a polypeptide of a plant-derived DNA demethylase having catalytic activity in DNA demethylation, or comprising a polynucleotide encoding the polypeptide.

2. The composition of claim 1, wherein the plant-derived polypeptide is Arabidopsis thaliana-derived DNA demethylase.

3. The composition of claim 1, wherein the polypeptide is DEMETER (DME) or a variant thereof.

4. The composition of claim 3, wherein the DEMETER variant has a deletion of an amino acid sequence at positions 1 to 677 from the N-terminus of an amino acid sequence of DME of SEQ ID NO: 1.

5. The composition of claim 3, wherein the DEMETER variant consists of an amino acid sequence of SEQ ID NO: 2.

6. The composition of claim 1, wherein the polypeptide is a demethylase that excises methylated cytosine from DNA.

7. The composition of claim 1, wherein the animal cell is a cell derived from a human, a cow, a horse, a pig, a dog, a sheep, a goat, a cat, or a mouse.

8. The composition of claim 1, wherein the interferon is selected from the group consisting of interferon-alpha (IFNα), interferon-beta (IFNβ), interferon-gamma (IFNγ), interferon kappa (IFN-κ), interferon-delta (IFN-δ), interferon epsilon (IFN-ε), interferon tau (IFN-τ), interferon omega (IFN-ω), and interferon zeta (IFN-ζ).

9. The composition of claim 1, wherein the composition is for in vitro, in vivo, or ex vivo administration.

10. An antiviral composition comprising a plant-derived polypeptide having catalytic activity in DNA demethylation or comprising a polynucleotide encoding the polypeptide.

11. A kit for increasing an expression level of interferon in animal cells, the kit comprising a plant-derived polypeptide having catalytic activity in DNA demethylation or comprising a polynucleotide encoding the polypeptide.

12. A method of increasing an expression level of interferon in animal cells, the method comprising introducing a polynucleotide encoding a plant-derived polypeptide having catalytic activity in DNA demethylation into the animal cells by incubating the polynucleotide with the animal cells.

13. The method of claim 12, wherein the method induces expression of an interferon gene through DNA demethylation of the animal cells.

14. The method of claim 12, wherein the method induces antiviral responses.


SEQUENCE LISTING (5 sequences)

SEQ ID NO: 1 - protein, 1729 aa, Artificial Sequence
  DEMETER protein [Arabidopsis thaliana]
MQSIMDSSAVNATEATEQNDGSRQDVLEFDLNKTPQQKPSKRKRKFMPKVVVEGKPKRKPRKPAELPKVVVEGKPKRKPRKAATQEKVKSKETGSAKKKNLKESATKKPANVGDMSNKSPEVTLKSCRKALNFDLENPGDARQGDSESEIVQNSSGANSFSEIRDAIGGTNGSFLDSVSQIDKTNGLGAMNQPLEVSMGNQPDKLSTGAKLARDQQPDLLTRNQQCQFPVATQNTQFPMENQQAWLQMKNQLIGFPFGNQQPRMTIRNQQPCLAMGNQQPMYLIGTPRPALVSGNQQLGGPQGNKRPIFLNHQTCLPAGNQLYGSPTDMHQLVMSTGGQQHGLLIKNQQPGSLIRGQQPCVPLIDQQPATPKGFTHLNQMVATSMSSPGLRPHSQSQVPTTYLHVESVSRILNGTTGTCQRSRAPAYDSLQQDIHQGNKYILSHEISNGNGCKKALPQNSSLPTPIMAKLEEARGSKRQYHRAMGQTEKHDLNLAQQIAQSQDVERHNSSTCVEYLDAAKKTKIQKVVQENLHGMPPEVIEIEDDPTDGARKGKNTASISKGASKGNSSPVKKTAEKEKCIVPKTPAKKGRAGRKKSVPPPAHASEIQLWQPTPPKTPLSRSKPKGKGRKSIQDSGKARGPSGELLCQDSIAEIIYRMQNLYLGDKEREQEQNAMVLYKGDGALVPYESKKRKPRPKVDIDDETTRIWNLLMGKGDEKEGDEEKDKKKEKWWEEERRVFRGRADSFIARMHLVQGDRRFSPWKGSVVDSVIGVFLTQNVSDHLSSSAFMSLAARFPPKLSSSREDERNVRSVVVEDPEGCILNLNEIPSWQEKVQHPSDMEVSGVDSGSKEQLRDCSNSGIERFNFLEKSIQNLEEEVLSSQDSFDPAIFQSCGRVGSCSCSKSDAEFPTTRCETKTVSGTSQSVQTGSPNLSDEICLQGNERPHLYEGSGDVQKQETTNVAQKKPDLEKTMNWKDSVCFGQPRNDTNWQTTPSSSYEQCATRQPHVLDIEDFGMQGEGLGYSWMSISPRVDRVKNKNVPRRFFRQGGSVPREFTGQIIPSTPHELPGMGLSGSSSAVQEHQDDTQHNQQDEMNKASHLQKTFLDLLNSSEECLTRQSSTKQNITDGCLPRDRTAEDVVDPLSNNSSLQNILVESNSSNKEQTAVEYKETNATILREMKGTLADGKKPTSQWDSLRKDVEGNEGRQERNKNNMDSIDYEAIRRASISEISEAIKERGMNNMLAVRIKDFLERIVKDHGGIDLEWLRESPPDKAKDYLLSIRGLGLKSVECVRLLTLHNLAFPVDTNVGRIAVRMGWVPLQPLPESLQLHLLELYPVLESIQKFLWPRLCKLDQRTLYELHYQLITFGKVFCTKSRPNCNACPMRGECRHFASAYASARLALPAPEERSLTSATIPVPPESFPPVAIPMIELPLPLEKSLASGAPSNRENCEPIIEEPASPGQECTEITESDIEDAYYNEDPDEIPTIKLNIEQFGMTLREHMERNMELQEGDMSKALVALHPTTTSIPTPKLKNISRLRTEHQVYELPDSHRLLDGMDKREPDDPSPYLLAIWTPGETANSAQPPEQKCGGKASGKMCFDETCSECNSLREANSQTVRGTLLIPCRTAMRGSFPLNGTYFQVNELFADHESSLKPIDVPRDWIWDLPRRTVYFGTSVTSIFRGLSTEQIQFCFWKGFVCVRGFEQKTRAPRPLMARLHFPASKLKNNKT

SEQ ID NO: 2 - protein, 673 aa, Artificial Sequence
  DME deltaN677 deltaIDR1::lnk
YKGDGALVPYESKKRKPRPKVDIDDETTRIWNLLMGKGDEKEGDEEKDKKKEKWWEEERRVFRGRADSFIARMHLVQGDRRFSPWKGSVVDSVIGVFLTQNVSDHLSSSAFMSLAARFPPAGSSGNGSSGNGTSQWDSLRKDVEGNEGRQERNKNNMDSIDYEAIRRASISEISEAIKERGMNNMLAVRIKDFLERIVKDHGGIDLEWLRESPPDKAKDYLLSIRGLGLKSVECVRLLTLHNLAFPVDTNVGRIAVRMGWVPLQPLPESLQLHLLELYPVLESIQKFLWPRLCKLDQRTLYELHYQLITFGKVFCTKSRPNCNACPMRGECRHFASAYASARLALPAPEERSLTSATIPVPPESFPPVAIPMIELPLPLEKSLASGAPSNRENCEPIIEEPASPGQECTEITESDIEDAYYNEDPDEIPTIKLNIEQFGMTLREHMERNMELQEGDMSKALVALHPTTTSIPTPKLKNISRLRTEHQVYELPDSHRLLDGMDKREPDDPSPYLLAIWTPGETANSAQPPEQKCGGKASGKMCFDETCSECNSLREANSQTVRGTLLIPCRTAMRGSFPLNGTYFQVNELFADHESSLKPIDVPRDWIWDLPRRTVYFGTSVTSIFRGLSTEQIQFCFWKGFVCVRGFEQKTRAPRPLMARLHFPASKLKNNKT

SEQ ID NO: 3 - protein, 10 aa, Artificial Sequence
  nuclear localization signal
PKKKRKVGVD

SEQ ID NO: 4 - protein, 12 aa, Artificial Sequence
  Linker
AGSSGNGSSGNG

SEQ ID NO: 5 - DNA, 35 bp, Artificial Sequence
  35 mer oligonucleotide
  modified_base (18): m5c
ctatacctcctcaactccggtcaccgtctccggcg